(19) United States
(12) Patent Application Publication
(10) Pub. No.: US 2026/0108184 A1
(43) Pub. Date: Apr. 23, 2026

(54) SYSTEMS AND METHODS FOR WEARABLE SENSING PLATFORM

(71) Applicant: Lume Health Technologies Inc., San Francisco, CA (US)

(72) Inventors: Jonathan Moustakis, San Francisco, CA (US); Victoria Meredith Powell, San Francisco, CA (US); Hang Yuan, Oxford (GB); Sneha Mukherjee, San Francisco, CA (US); Andrei Lvovch Gindilis, Mukilteo, WA (US); Timothy Michael Johnson, Sarasota, FL (US)

(21) Appl. No.: 19/153,703

(22) PCT Filed: Feb. 26, 2025
(86) PCT No.: PCT/US2025/017304

§ 371 Date: Aug. 5, 2025

Related U.S. Application Data
(60) Provisional application No. 63/558,545, filed on Feb. 27, 2024.

(51) Int. Cl. A61B 5/145; A61B 5/00; A61B 5/0205; A61B 5/024; A61B 5/11; A61B 5/1477; A61B 10/00

(52) U.S. Cl. CPC A61B 5/14521; A61B 5/0205; A61B 5/14546; A61B 5/1477; A61B 5/4857; A61B 5/486; A61B 5/6804; A61B 5/681; A61B 5/7278; A61B 10/0064; A61B 5/02405; A61B 5/1118; A61B 5/4806

(57) ABSTRACT

A wearable device may include a biofluid generation unit, a microfluidic channel for transporting the biofluid, and a sensing module including one or more electrochemical aptamer-based sensors having attached reporters that are displaceable by a target analyte. A wearable device may include electrodes for producing a potential sufficient for exchanging charged particles with the displaced reporters. A wearable device may include a processor for executing computer readable instructions including causing 2025/184151 activation of the electrodes to exchange the charged particles with the displaced reporters, receiving a measurement of the exchanged charged particles, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

### CROSS-REFERENCE TO RELATED APPLICATION

This application claims the priority benefit of U.S. Provisional Patent Application Ser. No. 63/558,545, filed Feb. 27, 2024, the contents of which are herein incorporated by reference in their entirety.

### INCORPORATION BY REFERENCE

All publications and patent applications mentioned in this specification are herein incorporated by reference in their entirety, as if each individual publication or patent application was specifically and individually indicated to be incorporated by reference in its entirety.

### TECHNICAL FIELD

This disclosure relates generally to the field of wearable biosensors, and more specifically, to the field of real-time measurement of biomarkers in a biological fluid.

### BACKGROUND

Circadian rhythm imbalances affect millions globally with consequences such as sleep disorders, chronic fatigue syndrome, and increased risks of diabetes, obesity, cancer, and cardiovascular diseases. Circadian rhythms can be determined through the measurement of biomarkers such as hormones and metabolites.

Melatonin, for example, is a hormone produced by the pineal gland and is the gold-standard marker of the central circadian clock located in the suprachiasmatic nucleus of the hypothalamus. The central circadian clock regulates almost all physiological processes including, but not limited to, the sleep-wake cycle, metabolism, cardiovascular functioning, and the gastrointestinal system. Circadian rhythms, measured through melatonin for example, play a crucial role in synchronizing biological processes with environmental cues such as light and darkness. Thus, disturbances in melatonin levels have been linked to cardiovascular diseases, cancer risk, metabolic disease, sleep disorders, mood disorders, and other health issues.

### SUMMARY

In some aspects, the techniques described herein relate to a non-invasive wearable device including: a biofluid generation unit configured to generate, or stimulate the generation of, a biofluid, wherein the biofluid generation unit includes at least one sweat generating hydrogel, and a muscarinic receptor agonist; a microfluidic channel configured to transport the biofluid, wherein the microfluidic channel includes an input end configured to receive the biofluid and an output end; a sensing module in fluid communication with the output end of the microfluidic channel and including one or more electrochemical aptamer-based sensors, wherein the one or more electrochemical aptamer-based sensors are functionalized with aptamers including attached reporters that are displaceable by a target analyte; electrodes configured to produce a potential sufficient for exchanging charged particles with the displaced reporters, or with conjugates of the displaced reporters, wherein the displaced reporters are configured to be hybridized with an enhancer to amplify a signal generated by the displaced reporters; and a processor communicatively coupled to the sensing module, the biofluid generation unit, the electrodes, and a memory configured to store computer readable instructions, wherein the processor is configured to execute the computer readable instructions including: causing activation of the electrodes to exchange the charged particles with the displaced reporters, or with conjugates of the displaced reporters, receiving a measurement of the exchanged charged particles, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

In some aspects, the techniques described herein relate to a computer-implemented method for generating individualized circadian entraining factors, the method including: receiving a measurement of a target analyte in a biofluid from an electrochemical aptamer-based sensor; determining a concentration of the target analyte in the biofluid based on the received measurement; detecting a circadian rhythm based on the determined concentration; and generating an individualized set of circadian entraining factors based on the detected circadian rhythm to guide a user towards an improved circadian amplitude.

In some aspects, the techniques described herein relate to a non-invasive wearable device including: an electrochemical, aptamer-based sensing device configured to sense and measure a target analyte in a biofluid; and a processor communicatively coupled to the electrochemical, aptamer-based sensing device, and a memory configured to store computer readable instructions, wherein the processor is configured to execute the computer readable instructions including: causing activation of the electrodes to exchange charged particles with displaced reporters, or with conjugates of the displaced reporters, receiving a measurement of the exchanged charged particles, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

In some aspects, the techniques described herein relate to a non-invasive wearable device including: a sensing module configured to sense and measure a target analyte in a biofluid; and a processor communicatively coupled to the sensing module, and a memory configured to store computer readable instructions, wherein the processor is configured to execute the computer readable instructions including: receiving a measurement of the target analyte in the biofluid, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 illustrates an example system for determining and managing a circadian rhythm of an individual.

FIG. 2A illustrates a block diagram of an example biomarker sensing device.

FIG. 2B illustrates a block diagram of an example monitoring application.

FIG. 2C illustrates a schematic drawing of an example partial graphical user interface.

FIG. 3A illustrates a schematic drawing of an example biomarker sensor integrated into the biomarker sensing device described herein.

FIG. 3B illustrates a schematic drawing of an example electrode configuration for the biomarker sensing device described herein.

FIG. 3C illustrates a schematic drawing of an example electrode configuration for the biomarker sensing device described herein.

FIGS. 4A-4D illustrate block diagrams representing example binding scenarios for at least one aptamer.

FIG. 5A illustrates a graphical diagram depicting an example change in electrical state caused by an electron exchange.

FIG. 5B illustrates a graphical diagram depicting detection of melatonin in artificial sweat by an aptamer functionalized with a redox reporter.

FIG. 6 illustrates a schematic of an example biomarker sensing device.

FIG. 7 illustrates an example flow diagram of an example process of generating individualized circadian rhythm recommendations.

FIG. 8 illustrates an example flow diagram of an example process of generating individualized circadian entraining factors.

The illustrated embodiments are merely examples and are not intended to limit the disclosure. The schematics are drawn to illustrate features and concepts and are not necessarily drawn to scale.

### DETAILED DESCRIPTION OF THE DRAWINGS

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

The measurement of biomarkers, such as hormones and metabolites related to circadian rhythm, may be used to determine an individual's circadian rhythm. Such measurements, however, are typically confined to laboratory settings and use invasive procedures and/or specialized equipment. Moreover, such biomarkers may be present in low concentrations and/or require hourly and/or overnight sampling, further complicating measurement processes.

The devices and methods described herein provide technical solutions to the above technical problems. The devices described herein include wearable devices that can noninvasively measure and/or monitor (over time) one or more hormones or metabolites without disturbing a user. Further, the devices and methods described herein employ signal amplification methodologies (e.g., including the aptamer-reporter assembly in microfluidic channel allowing for dissociation and reassociation of the target analytes to multiple aptamer reporter complexes; modification of the electrodes such as the inclusion of self-assembled monolayer consisting of 6-Mercapto-1-hexanol) to measure and analyze analytes in low concentrations. This technology enables the detection of analytes with both low molecular weight and extremely low abundance in sweat. This technology can also be adapted towards multiplexed sensing by housing different aptamers specific to different target analytes in the microfluidic channel.

Further, the devices and methods described herein may be practically applied in the field of individualized biometrics, and more specifically, the field of individualized circadian rhythm monitoring and adjustment. For example, the devices and methods described herein may be practically applied to tracking fertility and inflammation changes to support family planning and perimenopause management, assessing hormonal imbalances to guide hormone replacement therapy, and improving gut health. The technology can aid in identifying and managing chronic fatigue syndrome, diagnosing and treating sleep and circadian rhythm disorders, and monitoring major neurocognitive disorders such as Alzheimer's disease. Furthermore, the devices and methods can facilitate therapeutic drug monitoring to ensure drug levels remain within the optimal therapeutic range for conditions such as cancer, diabetes, infections, mental health disorders, thyroid dysfunction, and other hormonal imbalances.

The devices and methods described herein may be applied to a wide variety of hormones, analytes, biomarkers, and the like. For example, the devices and methods may be used to determine the concentration of biomarkers such as melatonin, cortisol, interleukin 6, neuropeptide Y, caffeine, glucose, lactate, ammonium, zinc, magnesium, estrogens, progesterone, testosterone, growth hormone, adenosine, serotonin, creatine kinase, insulin, glucagon, c-reactive protein, procalcitonin, leptin, ghrelin, thyroxin, ethanol, urea, uric acid, vitamin C, and vitamin D.

As used herein, the term biomarker refers to a molecule whose presence and/or concentration characterizes a biological condition. As described and exemplified herein, the sensing module 2080 is configured to sense melatonin as a biomarker for circadian rhythm. It should be understood, however, that the sensing module 2080 is not so limited and may be configured to sense other biomarkers, such as hormones and metabolites, that inform circadian rhythm, and/or molecules such as cortisol, interleukin-6, neuropeptide Y, caffeine, glucose, lactate, ammonium, zinc, magnesium, estrogens, progesterone, testosterone, growth hormone, adenosine, serotonin, creatine kinase, insulin, glucagon, c-reactive protein, procalcitonin, leptin, ghrelin, thyroxin, ethanol, urea, uric acid, vitamin C, and vitamin D, the presence and/or concentrations of which may assist in better determining or refining circadian rhythm.

In some examples described herein, the biomarker sensed by the sensing module may be referred to as an analyte or target analyte.

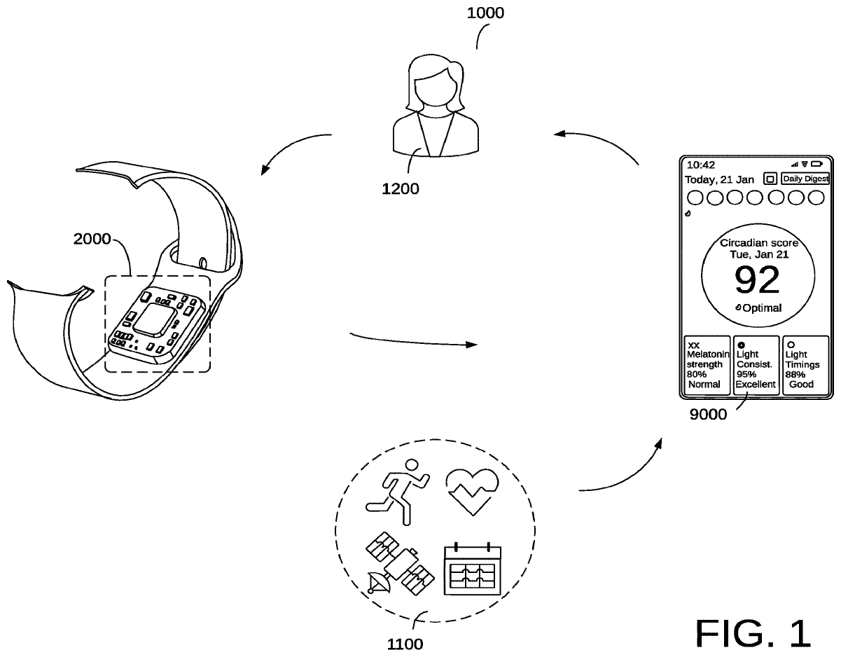

With reference to FIG. 1, a system 1000 for determining (e.g., detecting) and managing a circadian rhythm of an individual 1200 is shown and described. The system 1000 includes a non-invasive, biomarker sensing device 2000. The device 2000 may be a wearable device. The device 2000 may be positionable adjacent to a skin surface of an individual 1200. In some embodiments, the device 2000 may have one or more straps, may be a patch, or may otherwise be couplable to a skin surface of an individual 1200. For example, device 2000 may include a housing that at least partially defines a surface configured to adhere to a skin surface of a user. The device may be wearable on a wrist, a finger, a leg portion, an ankle, a foot (e.g., an arch, a foot pad, etc.), an arm portion, a forehead, a torso portion, a back portion, etc. The system 1000 may optionally include a monitoring application 9000 stored in memory on, and executed by a processor of, a communicatively coupled computing device. Alternatively, the monitoring application 9000 may be stored in memory on, and executed by a processor of, device 2000. In some embodiments, the system 1000 may further include one or more optional (shown by dashed lines) auxiliary input devices 1100 in the form of heart rate sensors, heart rate variability (HRV) sensors, light sensors, actigraphy sensors, global positioning system (GPS)/location sensors, map services, date/time devices, calendars, phone and app usage data, and the like, which may themselves be in a wearable form factor, embodied within a wearable device (e.g., a smartwatch), integrated with the biomarker sensing device 2000, or otherwise in communication with device 2000. Alternatively, or additionally, the system 1000 may receive data from one or more of heart rate sensors, HRV sensors, light sensors, actigraphy sensors, GPS/location sensors, map services, date/time devices, calendars, phone and app usage data, and the like using one or more Application Programming Interfaces (APIs).

In use, the biomarker sensing device 2000 is operable to stimulate the generation of a biofluid from the individual 1200 and analyze the biofluid to generate therefrom a signal correlated with a concentration of a target analyte in the biofluid. The data representing and/or derived from the signal may be transferred to a monitoring application 9000. The monitoring application 9000 can determine therefrom a circadian rhythm of the individual 1200, determine corrective actions (e.g., individualized circadian entraining factors) for presentation to the individual 1200 to correct disruptions in their circadian rhythm, and/or guide an individual 1200 to improve a circadian rhythm of the individual 1200.

In some embodiments, the monitoring application 9000 further receives auxiliary inputs from the one or more of the auxiliary input devices 1100 and uses the auxiliary inputs to refine its determination of the circadian rhythm, further analyze the circadian rhythm, and/or determine the corrective actions.

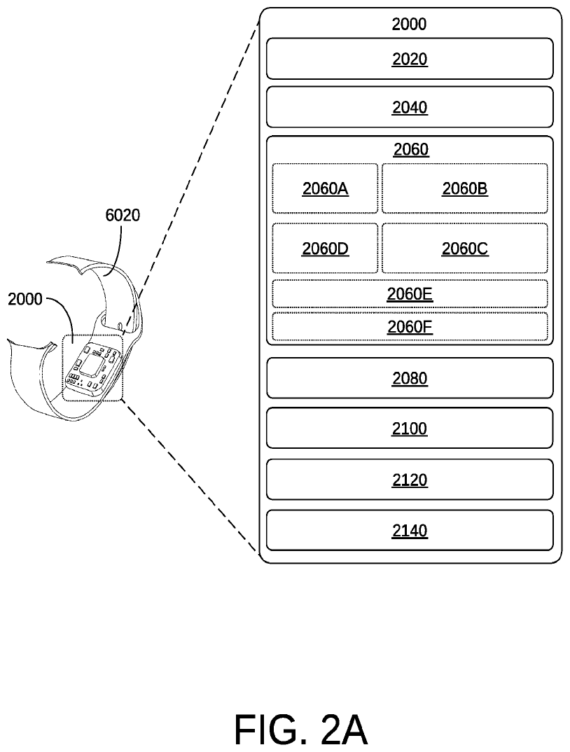

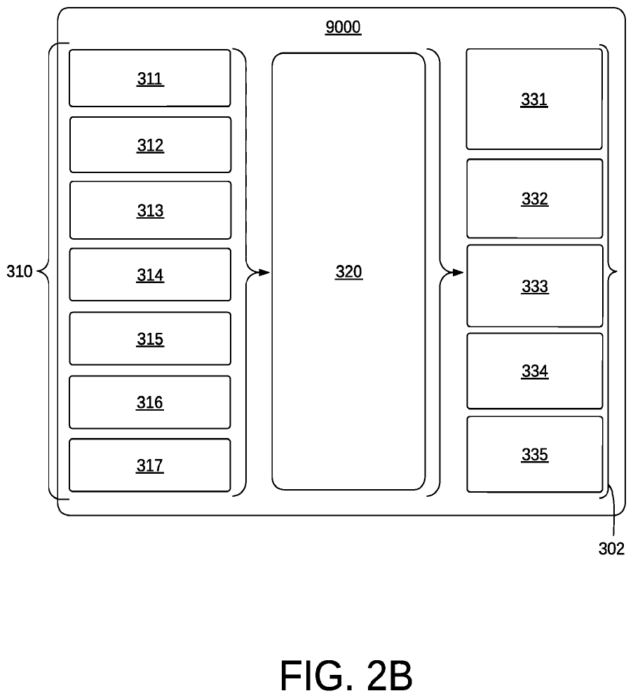

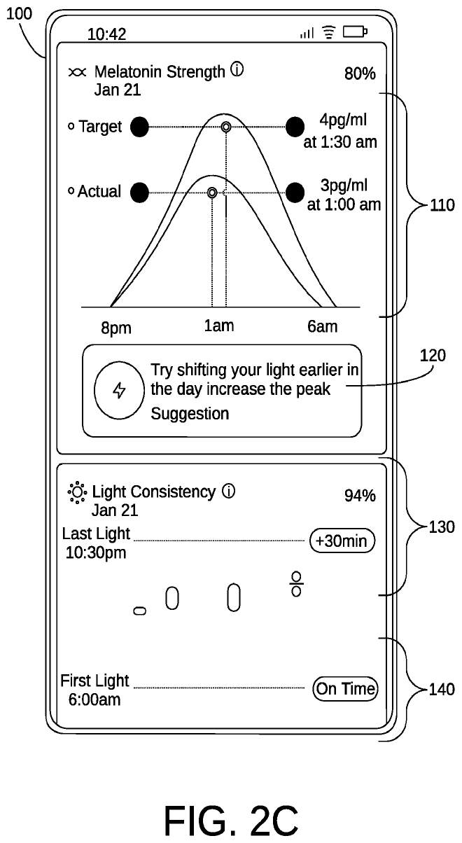

FIG. 2A illustrates a block diagram of an example biomarker sensing device 2000. In some embodiments, the biomarker sensing device 2000 is an electrochemical, aptamer-based (EAB) sensing device capable of capturing and/or monitoring molecular measurements (e.g., biomarker measurements). In some embodiments, the measurements are captured in real time. In some embodiments, the biomarker sensing device 2000 may interface with one or more other wearable devices, one or more server devices, and/or one or more databases to carry out analysis and communication of detected and generated data. In some embodiments, the biomarker sensing device 2000 is integrated into a strap 6020 that is wearable by the individual 1200 around their wrist, forearm, upper arm, torso, or other appropriate body part. In some embodiments, device 2000 is positionable to interface with at least a portion of a wrist of a user. The strap may be part of an external, wearable device such as that for a watch. In some embodiments, the strap 6020 has an integrated biomarker sensing device 2000. The biomarker sensing device 2000 may be positioned on the strap 6020 at a location where the biofluid generation unit 2020 of the biomarker sensing device 2000 is in firm contact with the skin of the individual 1200. In some embodiments of the strap-based physical form factor, the biofluid generation unit 2020 may be in firm contact with an underside of the wrist of the individual 1200. The strap 6020 may be removably engaged with the biomarker sensing device 2000, so as to be easily removed, replaced, or exchanged as desired. Other physical form factors that the biomarker sensing device 2000 can take include, but are not limited to, a wearable patch affixed to skin by adhesive; a device embedded within textile as clothing; a device affixed to spectacle arms using magnets or integrated into the spectacle arms; a device integrated into a headband, head strap, or headphones; a device integrated into a ring, necklace, or an electronic tattoo; a device integrated into an insole of a shoe, or a sock, and the like.

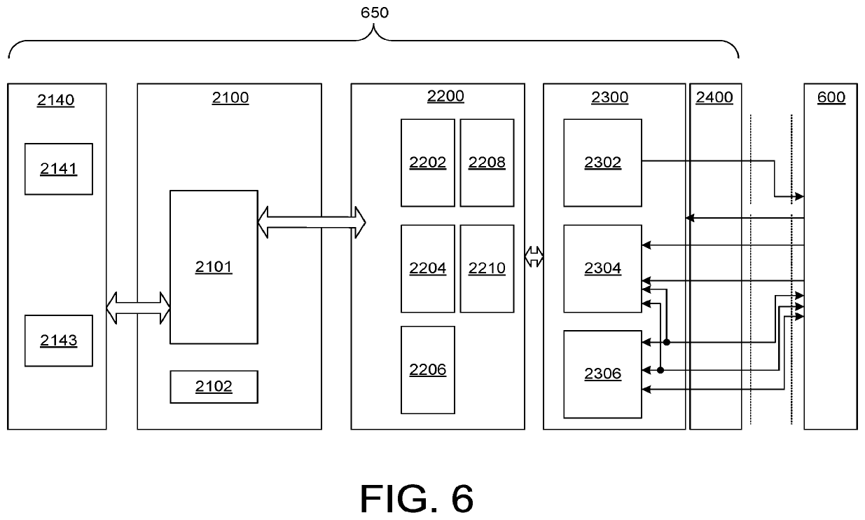

As schematically illustrated in FIGS. 2A and 6, the biomarker sensing device 2000 includes a biofluid generation unit 2020, a microfluidic channel 2040, a sensing module 2080, a signal processing unit 2100, a data transfer unit 2120, and a power source 2140. The biofluid generation unit 2020 may generate, or stimulate the generation of, a biofluid. The biofluid generation unit 2020 may include a sweat generating hydrogel. The biofluid generation unit 2020 may include a muscarinic receptor agonist. The biofluid generation unit 2020 may include an iontophoresis-based, sweat stimulator that utilizes electrical current to stimulate sweat in a particular body portion undergoing measurement. In some embodiments, the biofluid generation unit 2020 may alternatively be, or additionally include, an interstitial fluid, blood, or plasma collector and/or stimulator.

The microfluidic channel 2040 includes at least an input end and at least an output end. Either or both the input end or output end may be coupled to another component of the system, as described elsewhere herein. In some embodiments, the microfluidic channel 2040 may include one or more valves to prevent flow during periods of sensing by the sensing module 2080, for example. In some embodiments, the microfluidic channel 2040 does not include valves. In some embodiments, the microfluidic channel 2040 can also house the sensing module 2080 where the biorecognition elements may be embedded within the microfluidic channel 2040. In some embodiments, the microfluidic channel 2040 can also be functionalized to host the aptamer-reporter complex. In some embodiments, there may also be one or more reservoirs attached to the microfluidic channel 2040. These reservoirs may contain solutions to regenerate the reporters, add enhancers, or add buffer(s), as needed.

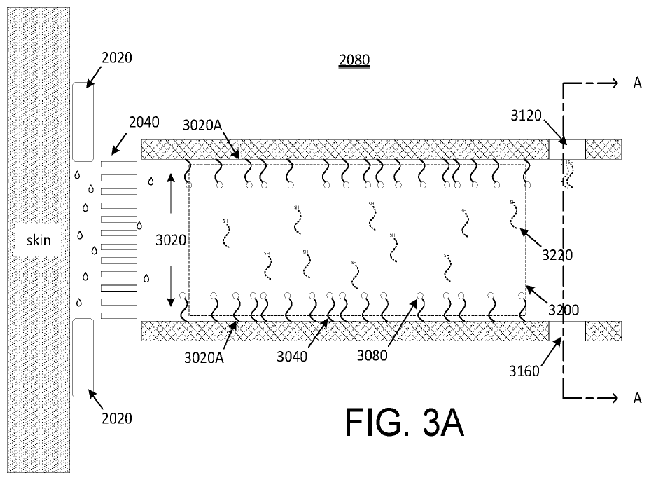

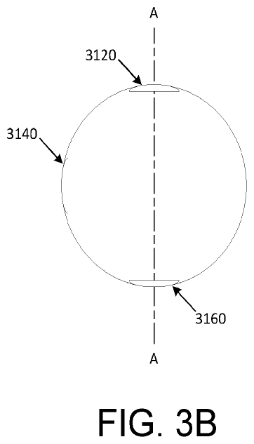

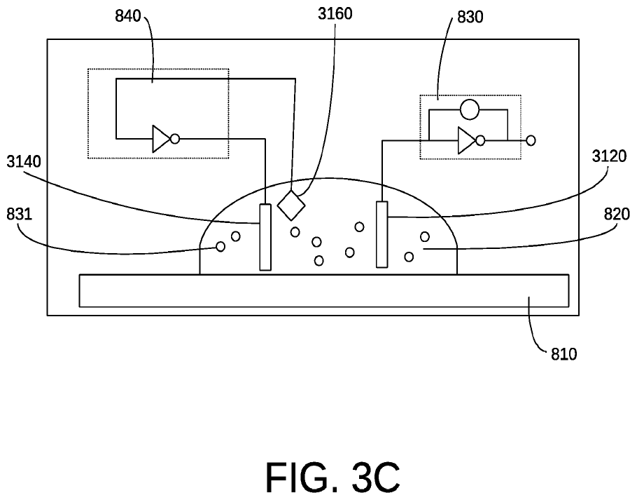

The sensing module 2080 may include one or more sensors for measuring, detecting, and/or monitoring biomarkers. In some embodiments, the sensing module 2080 is an EAB sensor, which is described in greater detail herein, with reference to FIGS. 3A-3B.

The signal processing unit 2100 may include one or more processors 2101, one or more controllers, one or more digital signal processors, etc. For example, the signal processing unit 2100 may include one or more hardware processors, including microcontrollers, digital signal processors, an application specific integrated circuit (ASIC), a field programmable gate array (FPGA) or other programmable logic devices, discrete gate or transistor logic, discrete hardware components, or any combination thereof designed to perform the functions described herein and/or capable of executing instructions, such as instructions stored by memory of device 2000 or a communicatively coupled device. In some embodiments, the signal processing unit 2100 includes a lead processor 2101 for executing, for example, system management (e.g., power sequencing, fault detection, and/or power optimization), battery charging, one or more safety protocols, connectivity to a computing device, and/or higher order sensor algorithms. In some embodiments, the signal processing unit 2100 includes a follower processor 2101 for executing, for example, peripheral or lower-level system management (e.g., general purpose input output (GPIO) multiplexing, signal conditioning, and/or data buffering), signal processing, and/or sensor management. The signal processing unit 2100 may also execute instructions for performing communications between the device 2000 and one or more servers, sensors, or any number of other computing devices. In some embodiments, the signal processing unit 2100 may execute instructions programmed into the device 2000. In some embodiments, the signal processing unit 2100 may execute instructions stored in memory 2102 of device 2000. In some embodiments, the signal processing unit 2100 may execute one or more algorithms and/or processes (e.g., process 800) stored on device 2000 and/or provided to or requested by device 2000.

The data transfer unit 2120 may be a wireless module (e.g., a Bluetooth™ communication module) operable to transmit data to a monitoring application (e.g., monitoring application 9000) using the Bluetooth™ communications protocol or other wireless protocol. In some embodiments, the data transfer unit 2120 may alternatively, or additionally, be or include a Near Field Communication (NFC) module, Wi-Fi communication module, infrared (IR) communication module, cellular communication module, or a port for a hardwired connection.

The power source 2140 may include or connect to a battery or a port for connecting the device 2000 to a power supply, an external battery, a wall power adapter, or the like. In some embodiments, power source 2140 includes a battery 2141 and battery charger and/or a wireless charging interface 2143 (e.g., using inductive charging).

The analog support system 2200 may include functions to support the remainder of the sensor. For example, the analog support system 2200 may include an analog to digital converter or ADC 2202 which input(s) are multiplexed between the different sensors. Similarly, a digital to analog converter or DAC 2204 may be multiplexed between the different sensors to drive or set specific voltages or current source set points. Buffered mid-rail voltages and accurate voltage references 2206 may be present to support the various amplifiers, trans-impedance amplifiers, and current sources in the system. One or more DC-DC converters 2208 or linear power supplies 2210 may be present to power the analog sub-systems. The power supply for the iontophoresis system may be present to generate the necessary voltages for the analog current source for iontophoresis. Due to variations in contact impedance between the skin and the electrodes as well as physiological differences in the skin characteristics between different users, iontophoresis may use a fairly significant voltage to guarantee the targeted current. This power supply may be adjustable in order to minimize the power of the current source and optimize battery life.

Sensing subsystem 2300 may include an adjustable iontophoresis current source 2302; potentiostat 2306; and/or pH, ionic strength, and/or temperature measurement sub-systems 2304. These functions may be designed to be dedicated specific to their function or they may be multi-function blocks which are configurable based on the targeted function. The design may be discrete off the shelf integrated circuits (ICs) or application specific ICs or any combination thereof. The block diagram of FIG. 6 is exemplary and may vary based on the targeted biomarker.

In some implementations, there may be many different copies of the same sensor's electrodes placed at various locations within the microfluidic channel 2040. Rather than replicate the active circuitry for each of the sensors, analog switches in the signal multiplexing subsystem 2400 may be utilized to individually select specific sensor electrode pairs and triplets. This multiplexing may be located on the disposable portion 600 or the reusable portion 650, whichever makes sense for the implementation(s). For example, in some embodiments, it may make more sense to put the analog switches on the disposable portion 600 so that the physical interconnect (number of contacts) is dramatically reduced between the disposable portion 600 and the reusable portion 650. In other embodiments, the benefits of this might be outweighed by the added costs to the disposable portion 600. As the disposable portion 600 and reusable portion 650 are designed to be separated and handled, protection against electrostatic discharge (ESD) may be needed. Static discharge onto the signals between the disposable portion 600 and reusable portion 650 may permanently damage the disposable portion 600, reusable portion 650, or both. This protection may be located on either the disposable portion 600, reusable portion 650, or both but is included in this exemplar in the signal multiplexing subsystem 2400.

In some embodiments, as shown in FIG. 6 and described above, device 2000 may include a reusable portion 650 and a disposable portion 600. For example, the disposable portion 600 may include the biofluid generation unit 2020, the microfluidic channel 2040, the sensing module 2080, and optionally the signal amplification unit or an on-board processing unit 2100. The disposable portion 600 may include a patch and/or an adhesive surface.

In some embodiments, the reusable portion 650 may include electronics for sensor measurement controls including timing, gains, filtering, etc. The electronics may further be used to determine the best and/or most opportune time to sample the sensors. The electronics may include data storage of sample data and/or log data so that communications with an application 9000 can be controlled. The electronics may include processing and controlling the individual sensors, scheduling the sampling of the sensors for the best and/or most opportune times taking into consideration motion or other environmental influences. The electronics may also include power management and/or battery management.

In some embodiments, the reusable portion of device 2000 may be worn separately on the body or may be mounted in specially tailored clothing or on a back side of a watch band (adjacent a skin surface).

The disposable portion 600 may have a limited lifespan and need to be replaced. In some embodiments, the disposable portion 600 may include one or more temperature sensors 2060B (FIG. 2A) so that sensor data can be appropriately calibrated for temperature.

In some embodiments, the biofluid generation unit 2020 may iontophoretically deliver a muscarinic receptor agonist such as, but not limited to, carbachol, pilocarpine, acetylcholine, methacholine, muscarine, and oxotremorine. The delivery of the agonist causes local stimulation of eccrine sweat glands in the dermis of the individual 1200, beneath the biofluid generation unit 2020.

The agonist may be stored in a hydrogel layer (which may be made from a single type or a combination of, alginate, agarose, methylacrylic anhydride, hydroxyethyl methacrylate, acrylamide, p-styrene-bipyridine, polyethylene glycol, and/or other monomers) between the iontophoretic electrodes and the skin surface (i.e., epidermis).

In some embodiments, the biofluid generation unit 2020 stimulates the generation of sweat thermally, by producing heat to stimulate the sweat glands locally.

In a further variation, the biofluid generation unit 2020 stimulates the generation of sweat vibrationally, for example using haptic motors.

Hydrostatic pressure generated by the eccrine sweat gland is used to channel sweat from the epidermis of the individual 1200 into an input end of the microfluidic channel 2040. In some embodiments of the sensing module 2080, valves are provided in the microfluidic channel 2040 to prevent flow during periods of sensing by the sensing module 2080.

In another embodiment, the biofluid generation unit 2020 stimulates the generation of interstitial fluid (ISF) through a reverse iontophoresis process, or alternatively, by sampling through microneedles.

In some embodiments, the biomarker sensing device 2000 further includes a biofluid preprocessing unit 2060 for preprocessing measured signals obtained by device 2000. The biofluid preprocessing unit 2060, if present, may fluidly connect to the input end or to some portion of the microfluidic channel 2040 and may preprocess sweat to aid in the aptamer-based electrochemical detection of the biomarker/target analyte. The biofluid preprocessing unit 2060 may include, and may be operable to perform, one or more of the following:

1. pH sensing 2060A using ion-selective electrodes and electrochemical transduction to measure electrolyte concentrations and/or pH, for calibration use during signal processing.

2. Temperature sensing 2060B using thermistors to measure the temperature of the biofluid sample (e.g., the biofluid received by the biofluid preprocessing unit 2060), for calibration use during signal processing.

3. Biofluid hyper-concentration 2060C. Optional semi-permeable microfluidic channels may allow for the forward osmosis of water out of the biofluid sample to hyper-concentrate the biomarker in the sample. An electrolyte concentration before and after hyper-concentration may be measured to determine the degree of hyper-concentration, for calibration use during signal processing.

4. pH buffering and/or ionic maintenance systems 2060D may use passive diffusion of ions and/or salts across semipermeable membranes to correct pH and/or electrolyte as needed.

6. Skin impedance measurement 2060E may utilize 2 or 4-electrode systems to determine sweat rate, hydrogel integrity, hydrogel moisture content, and hydrogel-electrode skin contact.

7. Electrolyte correction 2060F may use salt inclusion by pumping concentrated electrolyte solution, housed in a reservoir, to mix with the biofluid in order to increase the ionic content as needed.

The sensing module 2080 may be fluidly connected to an output end of the biofluid preprocessing unit 2060, if present, or to the output end of the microfluidic channel 2040 if the biofluid preprocessing unit 2060 is not present. The sensing module 2080, which is described in greater detail below with reference to FIGS. 3A-3B, generates a detectable change in an electrical state (e.g. a change in current) upon sensing of a target analyte in the biofluid. The change in electrical state correlates with a concentration of a target analyte in the individual 1200 and is presented as an electrical signal which is received by the signal processing unit 2100.

The signal processing unit 2100 processes the signal received from the sensing module 2080 to generate data representing or derived from the signal. The data generated by the signal processing unit 2100 may include one or more of voltage values, current values, changes in voltage values, changes in current values, and other derivatives of such values. In addition to generating these data, the signal processing unit 2100 may be operable to perform filtering and amplification to reduce noise in the received signal and/or amplify the signal. The data generated by the signal processing unit 2100 may be provided to the data transfer unit 2120, for example, for further processing, use, or display by a monitoring application 9000.

FIG. 2B illustrates an example architecture and operation of a monitoring application 9000. The monitoring application 9000 functions to receive one or more inputs 310; process, at block 320, the one or more inputs; and generate one or more outputs 302. For example, the methods may further include analyzing melatonin data alongside user inputs, environmental factors (e.g., ambient light, temperature, sunrise and sunset times, and/or other parameters), and other physiological parameters (e.g., activity (through actigraphy), heart rate, heart rate variability, blood pressure, pulse oximetry, and/or other parameters) to optimize circadian rhythm alignment (e.g., the degree of synchrony between the circadian phase as determined by the onset (primarily) and offset of melatonin production and the desired sleep-wake schedule) and improve circadian amplitude (e.g., the strength of the circadian rhythm as determined by the peak amplitude of the melatonin profile (highest concentration measured during the night) and/or the cumulative amount of melatonin measured overnight (the area under the curve of the melatonin profile). For example, the monitoring application 9000, running on the wearable device, a computing device, or other remote device, may receive one or more inputs 300 that include, but are not limited to, biomarker data, data from one or more other wearable devices (e.g., using APIs), other application data (e.g., calendar to cater to the user's schedule and adjust recommendations, meal/caffeine tracking and calorie intake information to help improve the recommendations, app usage data to inform exposure to night time light and screen time, menstrual cycle tracking data, etc.), user input (e.g., desired sleep and wake timing, work and exercise schedules, health goals, dietary preferences and known allergies and intolerances, medication and supplements, caffeine consumption, smoking habits, stress indicators, lifestyle information, notification and interaction parameters, behavioural and subjective inputs which will be used to inform time and intensity of entraining factors), user characteristics (e.g., demographic details, biometric attributes, health status and lifestyle factors, living conditions such as day or night shifts worker and other personal goals and preferences will be used to inform time and intensity of entraining factors), environmental data (e.g. weather to adjust light intake goals, location services to determine sunrise and sunset and adjust light intake recommendations,), user experiments (e.g., to experiment with non-standard entraining factor recommendations to determine their impact on their circadian profile), etc. At block 320, the monitoring application 9000 may use statistical models, machine learning techniques, and/or large language models (LLMs) to process the one or more inputs 300. The processing techniques at block 320 may be used to generate sleep actionable insights or an intervention plan, for example, including, but not limited to, circadian rhythm phase estimation, circadian rhythm amplitude estimation, a circadian rhythm profile (e.g., circadian phase and amplitude estimation), a sleep quality insight, a sleep performance insight, a personalized circadian plan (e.g., including circadian entraining factors), and/or an intelligent circadian coach (e.g., using a chatbot based LLM).

For estimation of circadian rhythm, the phase, amplitude and profile can be directly derived, from the melatonin concentration measurement. Circadian rhythm profile is the graph created from plotting the sample-to-sample melatonin concentration against the time it was measured. Circadian rhythm amplitude can either be approximated from the peak melatonin concentration recorded from the previous cycle or the cumulative amount of melatonin measured over the previous cycle. Circadian rhythm phase is determined from the time of onset (primarily) and offset of melatonin production, which correlates to the increase and decrease in measured melatonin concentration from the previous cycle.

For sleep insights (e.g., quality and performance), classification or scoring models like Gradient Boosted Trees may be used to map raw sensor data (e.g., movement, heart rate, respiration rate) to a “sleep quality index.” The sleep quality index may be a composite score reflecting overall sleep quality that may be based on analyzed raw sensor data (e.g., movement, heart rate, respiration) and extracted key sleep features (e.g., efficiency, duration, wake time) which are used to generate the sleep quality index. Further, one or more inputs to generate the sleep quality index may be weighted or normalized. Additionally, or alternatively, machine learning algorithms that employ predictive analytics may be used to identify the most impactful factors leading to poor sleep quality (e.g., late-night screen time, inconsistent schedule, etc.).

By way of example, a supervised learning model might use user-reported sleep diaries (labeled “good” or “poor” sleep) plus wearable sensor data to learn patterns. Once trained, the supervised learning model may predict nightly sleep quality for the user, highlight potential issues (e.g., evening stress spikes), and/or recommend improvements (e.g., wind-down routines).

In some embodiments, a Long Short-Term Memory (LSTM) neural network-based approach may be used to track consecutive nights of poor sleep to predict a drop in next-day focus and reaction times. As a result, the LSTM may recommend an earlier bedtime or additional short naps to boost performance on critical workdays.

For a personalized circadian plan, reinforcement learning may be used. For example, the system may try different interventions (e.g., earlier bedtime, morning bright light therapy) and measure improvements in circadian alignment (i.e., synchronizing the circadian profile with the desired timing of sleep onset and offset), adjusting recommendations over time. Alternatively, or additionally, recommendation systems may be used that draw on historical data from a plurality of users. For example, an algorithm may recommend effective changes based on a user's unique circadian profile.

For example, a collaborative filtering approach (conventionally used in e-commerce) may learn that users with a similar sleep pattern who adopted a 15-minute walk in bright morning light saw improved circadian stability (i.e., consistency in timing of the onset of melatonin production and offset of melatonin production or, said another way, consistency of the circadian phase). The collaborative filtering approach recommends the same intervention to the new user and tracks outcomes (e.g., using sensor data, feedback, user reported outcomes, etc.).

For an intelligent circadian coach, conversation and natural language generation may be used. The LLM can interpret user questions about sleep or circadian rhythms and generate coherent, context-aware answers. For adaptive coaching, the system may integrate machine learning insights (e.g., phase estimates, amplitude, etc.) and translate them into user-friendly tips or plans using a chatbot interface. The LLMs may be fine-tuned with expert-labeled prompt-instruction pairs provided with the user's circadian rhythm profile and personal characteristics. The LLMs may be further optimized using reinforcement learning from human feedback to continuously be made more intelligent as the user starts to have more interactions with the application 9000.

For example, the user may input: “I've been having trouble falling asleep around 11 PM. What should I do?” The chatbot (LLM) may access the user's circadian data (indicating a late-shifted phase), identify a relevant strategy (such as light exposure in the morning, limiting screen time, or adjusting meal schedules), and provide a concise, personalized explanation.

In some embodiments, the sleep actionable insights or invention plan, also described herein as outputs 302, may be based on a calculated concentration of the biomarker and/or target analyte from the data received from the biomarker sensing device 2000.

In some embodiments, the signal processing unit 2100 may convert the transduced signal from the aptamer-based electrochemical sensor or other methods mentioned elsewhere herein to a real time melatonin concentration, utilizing features extracted from the transduction signal, and calibrated using outputs from the preprocessing system. In some embodiments, the signal processing may be augmented with supervised machine learning models based on algorithms such as Support Vector Machines, Logistic Regression, Naive Bayes, Neural Networks, K-nearest neighbor, and Random Forest.

For example, a melatonin profile (concentration of melatonin over time) of an individual may be used to calculate or estimate a circadian rhythm phase of the individual (e.g., dim-light melatonin onset determination using the variable threshold method or alternatively the fixed threshold method) and/or a circadian amplitude of the individual (i.e., maximum concentration of melatonin and area under the melatonin time series curve).

As shown in FIG. 2B, one or more personalized machine learning models, at block 320, may be used to generate an intervention plan, which may include one or more circadian entraining factors. For example, using melatonin data or a melatonin profile, inputs from the individual (e.g., calendar inputs, important events such as flights and sporting events, ingestion information such as caffeine and food intake), inputs from other applications (e.g., maps, calendar, location services), learned intervention response habits of the individual (e.g., interventions which have a higher probability of adherence being prioritized, and interventions which have a higher personal effectiveness being prioritized), and/or inputs from other wearable sensors (e.g., ambient light, heart rate and variability, actigraphy, etc.), a statistical learning model or machine learning algorithm may be used to generate an intervention plan, including one or more circadian entraining factors, to correct circadian dysrhythmia and increase circadian amplitude in the individual.

In some embodiments, a biomarker or target analyte variability may be analyzed based on the concentration or profile and the intervention plan (e.g., including one or more circadian entraining factors) may be updated based on the hormone variability.

In some functional embodiments, the circadian rhythm data can be used to assist in medication ingestion timing including timing chemotherapeutic agents (where the circadian phase may be used to direct medication timing for peak efficacy); timing cardiometabolic treatments (where the circadian phase may be used to direct medication timing for peak efficacy); timing jet lag treatment (where the circadian phase may be used to direct intervention timing for peak efficacy); monitoring circadian rhythm disorder (through monitoring circadian phase and amplitude); treatment of circadian rhythm disorder (where the circadian phase may be used to direct medication and intervention timing for peak efficacy); monitoring of mental health disorders (through monitoring circadian phase and amplitude) including bipolar type 1 and type 2, seasonal affective disorder, and major depressive disorder; monitoring and/or timing of chronotherapeutic agents (where the circadian phase may be used to direct medication timing for peak efficacy); monitoring of shift workers or other workers (through monitoring circadian phase and amplitude); and /r monitoring of major neurocognitive disorders (through monitoring circadian phase and amplitude) such as Alzheimer's disease.

In some embodiments, additional data (e.g., received from one or more communicatively coupled computing devices) may be used to update one or more circadian entraining factors. The additional data may include heart rate data, HRV data, sleep data, and/or actigraphy data. For example, additional data can include photoplethysmography (PPG) data which indicates blood volume changes in the microvascular bed of tissue. Using PPG, the processor can determine a heart rate, an oxygen saturation, and/or a stress level using a speed of the changing volume of blood. Another method for adding additional data to the intervention recommendation algorithm can include near-infrared spectroscopy (NIRS). NIRS penetrates deeper into tissues to measure oxygenation levels and can be used to monitor muscle health and brain activity. Another method for adding additional data to the intervention recommendation algorithm can include magnetic resonance sensing. Other methods for adding additional data to the intervention recommendation algorithm can include temperature sensing of the skin and environment to infer core body temperature; and surface plasma resonance to determine or estimate biomolecular interactions.

Using the historical data from the application 9000 and other data modalities, time-series machine learning models (which may be based on algorithms such as Support Vector Machines, Logistic Regression, Naive Bayes, Neural Networks, K-nearest neighbor, and Random Forest) may be used to provide continuous monitoring of the melatonin data to provide insights into the user profile and alert the user based on any changes that might be of concern.

In some embodiments, the application 9000 may also enable an individual to interact with the melatonin data directly using an LLM-based AI agent so that the individual can ask questions about the collected data after the AI agent has been fine-tuned or grounded for each individual user.

In some embodiments, a gateway for clinical use cases may also be available to directly transfer the data, in a HIPPA compliant manner, to a clinical database with linked health record(s). Alternatively, an API from the application 9000 may be used to allow the users to participate in clinical studies or use the application 9000 as part of a clinical treatment such as cognitive behavioural therapy for insomnia.

In some embodiments, application 9000 may trigger the generation of feedback based on data received after incorporating an intervention plan, for example including one or more circadian entraining factors. The application 9000 may trigger the generation of one or more reminders to encourage the individual to employ or adopt one or more circadian entraining factors or an intervention plan.

For training the models at block 320, large sets of data may be labeled (or partially labeled). The data sets may include sleep data, wearables data, biomarkers data (like melatonin), subjective reports, and/or outcomes (e.g., sleep quality, next-day alertness, etc.). The system learns by finding correlations and patterns that link circadian features to real-world measures of sleep performance or circadian alignment. The models (e.g., neural networks, ensemble methods, or other sophisticated models) can transform raw signals (e.g., time series data) into circadian metrics or recommendations. For example, the model may identify patterns or embeddings in sensor data that are not immediately obvious to humans. The models may be trained iteratively on historical data, while adjusting parameters (e.g., weights in a neural network) to minimize prediction errors. Performance may be validated on a subset of data not used during training, ensuring real-world generalizability. The outputs of the models may include, but not be limited to, quantitative estimates (e.g., circadian phase, amplitude, sleep quality score, projected performance, etc.), and/or recommendations and/or intervention plans (e.g., “shift your bedtime by 30 minutes,” “use bright light therapy,” or “incorporate a short nap.” etc.). In some embodiments, the models may function to deliver feedback through a user interface or chatbot that explains the reasoning in accessible language (e.g., leveraging an LLM).

By blending these modeling approaches-statistical time series, machine learning, and natural language generation-an advanced, personalized system emerges. Application 9000 not only provides robust circadian insights (e.g., phase, amplitude, sleep quality) but also proactively guides users using meaningful interventions and coaching, ultimately promoting better sleep and overall well-being.

FIG. 2C illustrates a schematic drawing of an example partial graphical user interface or GUI 100. In some embodiments, a processor of, or communicatively coupled to, a biomarker sensing device 2000 may update or generate a GUI that includes a circadian rhythm profile and/or other biomarker information relevant to an individual. For example, the GUI may present one or more recommendations for the individual. The GUI 100 may be presented on a display of the biomarker sensing device 2000 or on a display of a communicatively coupled computing device. In some embodiments, the GUI 100 may display an amount, concentration, level, and the like of a biomarker and/or analyte 110. For example, the biomarker and/or analyte may be displayed relative to time, one or more activities (e.g., sensed or input), a mealtime, a sleep time, exercise time, etc.

As shown in FIG. 2C, the application 9000 may display the measured melatonin or other biomarker in time-dependent diagrams to showcase its trajectories with days and trends over a longer time. Key statistics 110 such as peak biomarker level and timing may also be highlighted or displayed. The application 9000, which may receive data from device 2000 may store the raw biomarker profiles, processed circadian rhythm data (phase and/or amplitude), user preferences, user inputs, relevant data from other apps (mentioned elsewhere herein), data from other wearable devices (mentioned elsewhere herein), and/or other pertinent data in a secure cloud server to allow for historical look up by the user or locally on the device 2000 or a communicatively coupled device.

In some embodiments, GUI 100 may further display a light consistency metric 130 and/or a first light metric 140. The light consistency metric 130 may indicate consistency of light intake particularly the time of first and last light intake and/or be determined based on readings from a light sensor. The first light metric 140 may indicate time of first light intake above a predetermined intensity such as about 1000 lux and/or be determined based on user input. In some embodiments, GUI 100 may display or provide a sleep actionable insight and/or a circadian entraining factor 120. The circadian entraining factor 120 may provide a recommendation, guidance, or other actionable insight to encourage an individual to adjust their behavior to improve a circadian rhythm of the individual. For example, as shown in FIG. 2C, the circadian entraining factor 120 may highlight or indicate the use of light as a way to improve the circadian cycle using biomarker detection. In some embodiments, the circadian entraining factor 120 may include or recommend a timing of light exposure. In some embodiments, the circadian entraining factor 120 may include or recommend an intensity of light exposure. In some embodiments, the circadian entraining factor 120 may include or recommend a meal timing. In some embodiments, the circadian entraining factor 120 may include or recommend an exercise timing. In some embodiments, the circadian entraining factor 120 may include or recommend a sleep timing.

FIG. 3A illustrates a schematic drawing of an example sensing module 2080 integrated into the biomarker sensing device described herein. As illustrated in FIG. 3A, the sensing module 2080 includes a microfluidic channel 3020. The microfluidic channel 3020 has an input end for receiving a sample of the biofluid generated by the biofluid generation unit 2020, for example by way of the microfluidic channel 2040. The microfluidic channel 3020 can transport biofluid to the sensing module 2080.

In some embodiments, the microfluidic channel 3020 may be functionalized with aptamers 3040 that bind with the biomarker/target analyte(s). In the case of melatonin, the aptamers 3040 may have the sequence (5′-3′), ACTCTCGGGACGACGTCTTGGGGGTGGTGGGTTTGGCTGGTACTTAGGGCGTCGT CCC. The microfluidic channel 3020 may include within it an enhancer solution 3200 containing enhancers 3220. In some embodiments, the enhancer solution 3200 is pre-mixed with the biofluid sample by the biofluid preprocessing unit 2060 prior to the biofluid sample being received by the microfluidic channel 3020. In another embodiment, the microfluidic channel 3020 is pre-filled with the enhancer solution 3200.

For cortisol measurements, the aptamer sequence may be (5′-3′) CGA CCG GTC TGG GGA CCC TGT CTG GGT GTG TGG GTA GTA GGT CG. For testosterone measurements, the aptamer sequence may be (5′-3′) AAC ACG AGG ACA GTG AGC TAG GGT CTG TCG TGA GCA TGA CCC GCC CGC AGT GTT.

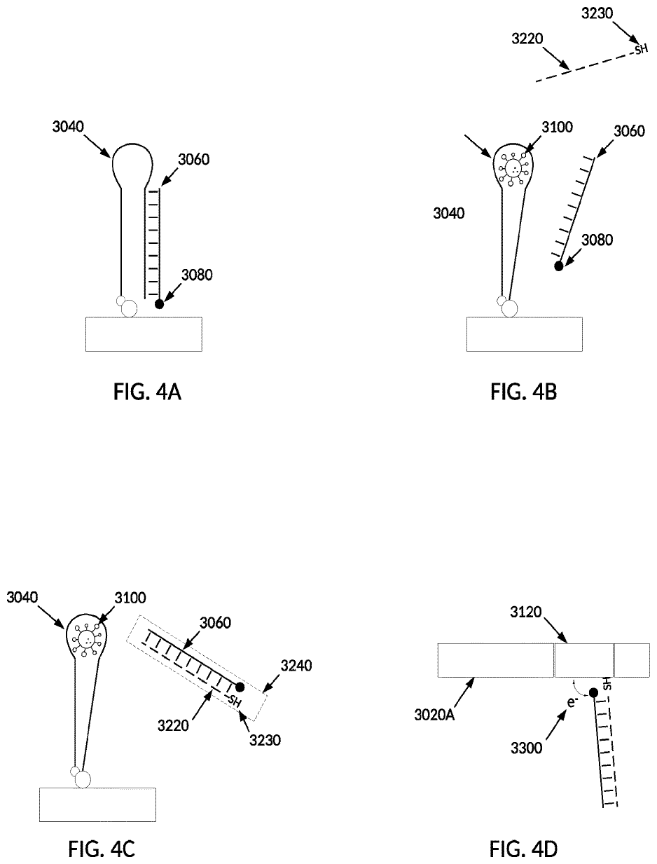

The aptamers 3040 are fixed to an inner surface 3020A of the microfluidic channel 3020 to functionalize the microfluidic channel 3020. In some embodiments, each aptamer 3040 is bound at their 5′end to a biotin molecule, which in turn binds to a streptavidin molecule that is bound to the inner surface 3020A (FIG. 4A). In another embodiment, the inner surface 3020A of the microfluidic channel 3020 is a gold-plated surface, and the aptamers are bound at their 5′end to the inner surface 3020A by a thiol bond. In a further embodiment, the 5′end the aptamers 3040 are instead bound to a bead or a metallic nanoparticle, such as a gold nanoparticle. In some embodiments, the inner surface 3020A is magnetized, or alternatively, a magnetic field is generated in the vicinity of the inner surface 3020A, to magnetically attract and fix, to the inner surface 3020A, the bead or metallic nanoparticle with the aptamers 3040 bound thereto.

Each aptamer 3040 is functionalized with a reporter 3060 (FIG. 4A). The reporter 3060 is functionalized with a redox tag 3080, at least in some embodiments, and is bound to the aptamer 3040. In some embodiments, the redox tag 3080 is Methylene Blue.

As further illustrated in FIGS. 4A to 4D, upon the aptamer 3040 binding with a target analyte 3100 (e.g. melatonin), the aptamer 3040 undergoes a conformational change which displaces the reporter 3060 and causes the reporter 3060 to detach (FIG. 4B) from the aptamer 3040. Upon detachment, the reporter 3060 hybridizes with an enhancer 3220 (FIG. 4C) in the enhancer solution 3200. Each enhancer 3220 is a complementary strand of the reporter and has a thiol molecule 3230 attached at its 5′end. Upon hybridization, the reporter 3060 and enhancer 3220 form a conjugate 3240, which diffuses towards a working electrode 3120 of the sensing module 2080 (FIG. 4D).

FIGS. 3B-3C illustrate schematic drawings of example electrode configurations for the biomarker sensing device 2000. The working electrode 3120 of the sensing module 2080 may be a gold-plated electrode. Conjugates 3240 bind to the working electrode 3120 by way of a bond between a molecule 3230 attached to the conjugate 3240 and a particle in the gold-plated electrode. For example, conjugates 3240 bind to the working electrode 3120 by way of a bond between a thiol molecule 3230 attached to the conjugate 3240 and gold particles in the gold-plated electrode.

Upon binding of the conjugate 3240 to the working electrode 3120, the redox tag 3080 attached to the reporter 3060 in the conjugate 3240 is brought to within a distance of the working electrode 3120 that facilitates electron exchange 3300 between the redox tag 3080 and the working electrode 3120, thereby generating a change in electrical state detectable by the working electrode 3120 (FIG. 4D). Said another way, the working electrode 3120 produces a potential sufficient for exchanging charged particles with the displaced reporters. The change in electrical state of a redox mediator at the working electrode 3120 is detected through use of Square Wave Voltammetry (SWV) methods, which are known to those in the art and is not further described herein.

Referring back to FIG. 3A and as also shown in FIG. 3C, the sensing module 2080 further includes a counter electrode 3140 and reference electrode 3160, which together with the working electrode 3120 form a three-electrode electrochemical cell well known to those in the art, and which is not further described herein.

As shown in FIG. 3C, the biomarker sensing device is in contact with at least a portion of a skin surface 810 to capture biomarkers 831 from biofluid 820 (e.g., sweat, interstitial fluid, etc.). The biomarkers 831 undergo a reaction with the aptamers, as described with respect to FIG. 3A and elsewhere herein and release a signal that can be electrochemically detected. The working electrode 3120 transmits signals to the detection unit 830. The detection unit 830 processes the signal, which may be amplified or converted into a readable format. The detection unit 830 may include an operational amplifier (op-amp) circuit, which may be used to process the electrical signals corresponding to the biomarker concentration. The feedback control unit 840 fine-tunes the operation of the sensing module by adjusting the applied voltage or current, optimizing sensitivity and stability. The feedback control unit 840 may further include a circuit for real-time adjustments based on feedback from the sensing module.

In some embodiments of the biomarker sensing device 2000, the microfluidic channel 3020 of the sensing module 2080 may receive a sample of the biofluid directly from the biofluid generation unit 2020, directly from the site at which the biofluid is produced (e.g. the surface of the skin), or otherwise without the need for the microfluidic channel 2040.

In a further variation of the biomarker sensing device 2000, the microfluidic channel 3020 may be functionalized with additional aptamers 3040 that are reactive to other molecules, such as cortisol, interleukin 6, neuropeptide Y, caffeine, glucose, lactate, ammonium, zinc, magnesium, estrogens, progesterone, testosterone, growth hormone, adenosine, serotonin, creatine kinase, insulin, glucagon, c-reactive protein, procalcitonin, leptin, ghrelin, thyroxin, ethanol, urea, uric acid, vitamin C, and vitamin D.

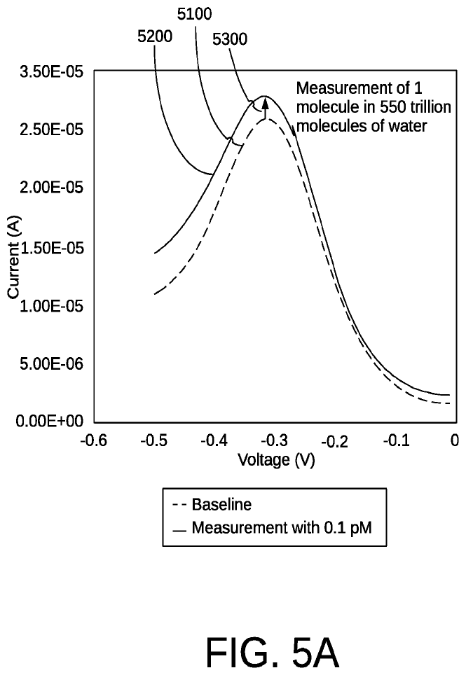

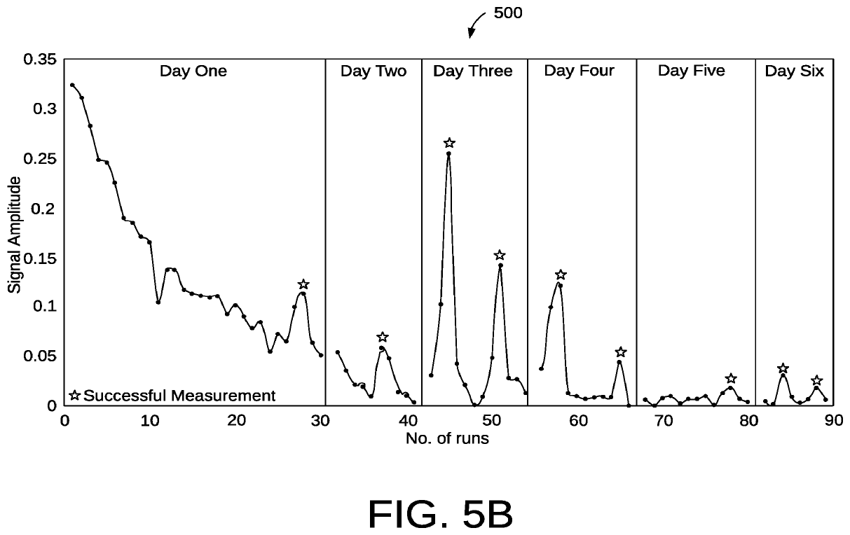

An exemplary change in electrical state caused by the electron exchange 3300 is illustrated in FIG. 5A. The dotted line represents a baseline current 5100 detected at the working electrode 3120 upon application of a stepped SWV voltage input in the absence of any biomarker/analyte. The solid line represents the detected current 5200 at the working electrode 3120 upon application of a stepped SWV voltage input in the presence of 0.1 pM of melatonin. The difference 5300 between the baseline current 5100 and the target-present or detected current 5200 is indicative of, and varies with, the concentration of melatonin.

The biomarker sensing device 2000 is in this manner is able to generate and detect a change in electrical state (e.g. difference in current) indicative of the concentration of a biomarker/analyte in the individual 1200 and transmit data representing or derived from this change in electrical state signal to the monitoring application 9000 for further processing and the determination of appropriate corrective actions (e.g., individualized circadian entraining factors).

The biomarker sensing device 2000 is more fully described by reference to the following non-limiting example.

FIG. 5B shows a graph 500 depicting a working example of detection of melatonin in artificial sweat by an aptamer functionalized with a redox reporter. Magnetic beads were used as a matrix to host the aptamer-reporter conjugates within a flow-through channel, situated either in capillary tubing or a microfluidic chip. The beads were functionalized with streptavidin, which secures the aptamer via streptavidin-biotin binding. Artificial sweat was circulated through the channel at a rate mimicking iontophoretic conditions, using a syringe pump. The artificial sweat, enriched with various concentrations of melatonin or a mix of enhancer and melatonin, was introduced into the channel. After a predetermined interval, the supernatant was collected from the channel's exit and analyzed using electrochemical techniques. As melatonin entered the channel, it bound to the aptamer, triggering a conformational shift that released the reporter, which was functionalized with methylene blue. The enhancer, possessing a sequence complementary to the reporter, bound to the newly released reporter and moved toward the channel outlet. The enhancer used in these experiments was modified with a thiol group, enabling it to form a strong bond with a gold electrode through a sulfur atom of the thiol. The bond facilitates the proximity of methylene blue to the electrode, thus enhancing electron transfer and generating a detectable electronic signal.

Each data point on the plot represents a discrete measurement. Initially, the signal amplitude decreased, indicating the clearance of unbound reporters. Once the baseline was achieved, the system received injections of melatonin at varying concentrations, marked by a significant rise in signal amplitude, denoted by a star symbol. After each data point, the system was returned to baseline by flushing with one or more buffers before introducing a new melatonin-spiked solution for subsequent measurements. This setup allowed for continuous monitoring over six days, although the signal intensity gradually diminishes as the channel depletes its supply of reporters.

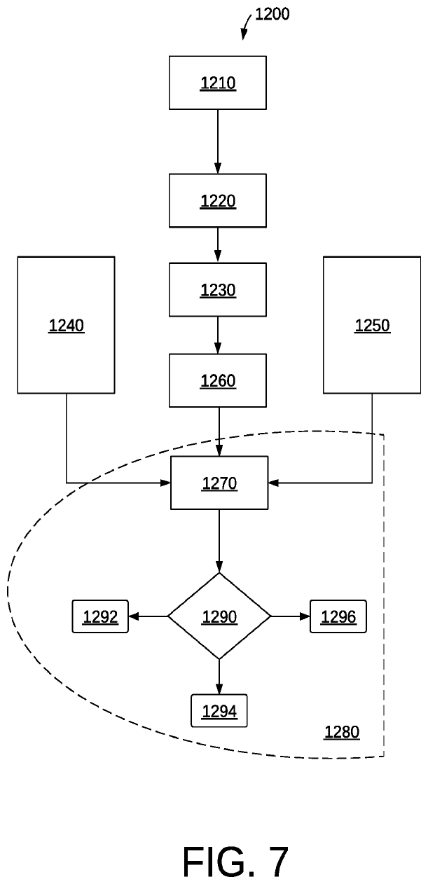

FIG. 7 illustrates an example flow diagram of an example process 1300 of generating individualized circadian rhythm recommendations. The flowchart illustrates a system that processes melatonin measurements to provide personalized recommendations for users to help improve their circadian rhythm. As shown in FIG. 7, electrochemical sensing 1210 includes detecting biomarker levels (e.g., melatonin) and/or electrolyte levels, sweat rate, body temperature and/or pH, etc. Relevant signal features are extracted from the detected signals, which are digitized using an ADC unit 1220 and analyzed by the signal processing unit 1230. The ADC unit 1220 unit converts the incoming analog signal into a digital format. This digital signal flows to the signal processing unit 1230, which refines and analyzes the data to determine a biomarker concentration 1260 of the individual. These data, which may be combined with additional inputs, such as wearable sensor information 1240 (e.g., sleep patterns, heart rate, exercise routines, meal timing, and light exposure) and/or contextual data 1250 (e.g., temperature, humidity, individual's schedule, daylight availability, user location, etc.), may undergo further processing 1270, for example using a statistical learning model or machine learning model as described above herein. The system may generate one or more GUIs 1280 of the processed data, offering insights into the user's biomarker concentration and associated factors. The system can synthesize inputs and personal parameters to generate individualized recommendations 1290 (e.g., entraining factor 1292, entraining factor 1294, entraining factor 1296, etc.). The individualized recommendations 1290 may be tailored to the individual's preferences (e.g., notification style, schedule, etc.) and/or needs (e.g., health conditions, lifestyle constraints, etc.). The individualized recommendations 1290, such as lifestyle adjustments or health tips, may be displayed through a GUI 1280 helping the user make informed decisions and improve their well-being based on real-time data and contextual factors relevant to their daily routines.

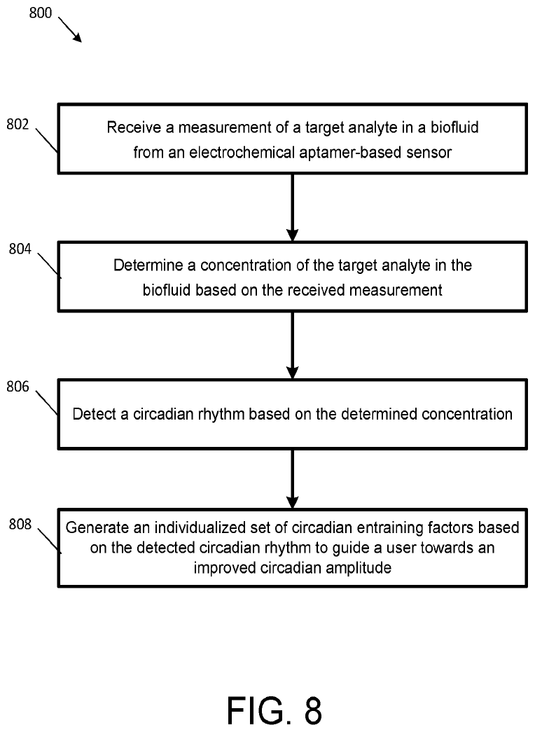

FIG. 8 illustrates an example flow diagram of an example process 800 of generating individualized circadian entraining factors. In general, the process 800 may be a computer-implemented method that may receive a measurement of a target analyte in a biofluid from an electrochemical aptamer-based sensor, determine a concentration of the target analyte in the biofluid based on the received measurement, detect a circadian rhythm based on the determined concentration, and generate an individualized set of circadian entraining factors based on the detected circadian rhythm to guide a user towards an improved circadian amplitude.

In some embodiments, the process 800 may be performed by a non-invasive wearable device (e.g., the signal processing unit 2100 and/or data transfer unit 2120 on biomarker sensing device 2000). In some embodiments, the process 800 may be performed by a server or cloud-based device. In some embodiments, the process 800 may be performed on a companion mobile computing device, such as within an app using a processor on a mobile phone. In some embodiments, the process 800 may be performed on a processor of the mobile computing device (e.g., as part of a native operating system on the mobile device and/or as stored as computer readable instructions in a memory of the mobile computing device). The process 800 may be carried out on other computing devices (e.g., laptops, tablets, or the like) in the event that sensor data is received from the device 2000 and performed by monitoring application 9000, for example.

In embodiments that utilize one or more direct sensor measurements, the sensor(s) may be part of a sensing module (e.g., sensing module 2080) that includes at least one electrochemical aptamer-based sensor device 2000. The electrochemical aptamer-based sensor device 2000 may be functionalized with aptamers including attached reporters. Further, the wearable device (e.g., non-invasive sensing device 2000) may include electrodes that may bear (e.g., produce) a potential sufficient for exchanging charged particles with the reporters displaced by a target analyte, or with conjugates of the displaced reporters. In such examples, each reporter may be displaceable by the target analyte and each displaced reporter may be hybridized with an enhancer to amplify a signal from the displaced reporters, as described elsewhere herein.

At block 802, the process 800 may include receiving a measurement of a target analyte in a biofluid from an electrochemical aptamer-based sensor. For example, a direct measurement from the sensor (e.g., biomarker sensing device 2000) or data representing the direct measurement may be received at a processing device. The measurement of the target analyte in the biofluid may be obtained from device 2000, for example, which may function to measure the target analyte (e.g., one or more biomarkers). In such an example, the device 2000 may include one or more sensors for capturing continuous molecular measurements for target analytes including, but not limited to melatonin, cortisol, testosterone, estrogen, growth hormone, thyroid hormones, interleukin 6, neuropeptides Y, caffeine, glucose, lactate, ammonium, zinc, magnesium, adenosine, serotonin, creatine kinase, insulin, glucagon, c-reactive protein, procalcitonin, leptin, ghrelin, thyroxin, ethanol, urea, uric acid, vitamin C, and vitamin D. In operation, the measurements may be obtained at the sensor(s) through sweat and/or interstitial fluid. Such measurements can be provided as input to the process 800. Alternatively, the measurements may be stored and/or transmitted to another computing device to carry out the process 800.

At block 804, the process 800 may include determining a concentration of the target analyte in the biofluid based on the received measurement. For example, the process 800 may include receiving measurement data captured by stimulating generation of a biofluid for a user. The biofluid may be analyzed to generate a signal correlated with a concentration of a particular target analyte present in the biofluid. For example, generating the signal may include performing square wave voltammetry on the processed biofluid and extracting the peak current. Optionally, analyzing the signal may include calibrating the peak current output of square wave voltammetry for temperature, ionic strength, sweat rate (if sweat is the biofluid), and other factors, and then using a calibration plot to convert that current amplitude to an analyte concentration.

At block 806, the process 800 may include detecting a circadian rhythm based on the determined concentration. For example, the process 800 may include performing (or receiving) sensor measurements monitored over a time period. The monitoring process may result in obtaining measurements with differing levels of analyte in each measurement. The different concentrations of analyte levels may be correlated to particular circadian rhythm amplitude changes over the time period. From the amplitude changes, the process 800 may determine a specific circadian rhythm for the user at a particular time period.

At block 808, the process 800 may include generating an individualized set of circadian entraining factors (e.g., entraining factor 120, entraining factor 1292, entraining factor 1294, entraining factor 1296, etc.) based on the detected circadian rhythm to guide a user towards an improved circadian amplitude. Example circadian entraining factors may include, but are not limited to, one or more of: a timing of light exposure, an intensity of light exposure, a meal timing, an exercise timing, or a sleep timing. The circadian entraining factors may be provided as output to a user on device 2000 and/or a mobile computing device and/or at a monitoring device associated with the user of device 2000, for example, as suggestions for improving circadian rhythm in the user. In operation, the process 800 may include all or a portion of process 1300 (FIG. 7) to generate one or more entraining factors.

In some embodiments, the process 800 further includes analyzing a hormone variability based on the determined concentration and updating the individualized set of circadian entraining factors based on the analyzed hormone variability. For example, sensitivity to light as a circadian entraining factor may be determined by determining the effect the default light recommendations have on a circadian rhythm of the individual. If the individual is sensitive to light, based on detected hormone variability, the individualized set of circadian entraining factors may be updated to recommend avoidance of nighttime light, for example, one hour earlier than the default. Further for example, if the individual is insensitive to light, the individualized set of circadian entraining factors may be updated to recommend increased morning and midday light in order to cause the intended effect on their circadian rhythm. If the individual has a low amplitude circadian rhythm, the individualized set of circadian entraining factors may be updated to recommend increased duration and/or intensity requirements for midday light. If the individual has a delayed circadian rhythm (i.e., the onset and peak melatonin production is later than normal for their sleep-wake schedule), the individualized set of circadian entraining factors may be updated to recommend an earlier and longer duration and more intense morning light exposure. If the individual has an advanced circadian rhythm (i.e., the onset and peak melatonin production is earlier than normal for their sleep-wake schedule), the individualized set of circadian entraining factors may be updated to recommend a later and longer duration and more intense morning light exposure.

In some embodiments, the process 800 further includes receiving data from one or more communicatively coupled remote devices (e.g., a mobile computing device, a wearable computing device, or a server). The data from the one or more communicatively coupled remote devices may include heart rate data, heart rate variability data, sleep data, or actigraphy data, and the like. The received data may be used to update the individualized set of circadian entraining factors.

In some embodiments, the process 800 further includes triggering generation of feedback based on data received after incorporating one or more of the individualized set of circadian entraining factors. For example, the application 9000 may trigger such feedback generation by analyzing updated physiological and behavioral data (e.g., acquired using device 2000, based on user inputs or reported outcomes, and/or based on data received from one or more other communicatively coupled devices) after implementing individualized circadian entraining factors. Based on this analysis, application 9000 provides personalized recommendations, alerts, or adjustments to optimize circadian alignment. These dynamic feedback mechanisms ensure tailored interventions, promoting improved circadian health and well-being.

In some embodiments, the process 800 further includes triggering generation of one or more reminders to employ the individualized set of circadian entraining factors. For example, the application 9000 may trigger generation of reminders to encourage the user to employ or adopt one or more circadian entraining factors or an intervention plan.

### EXAMPLES

Example 1. A non-invasive wearable device comprising: a biofluid generation unit configured to generate, or stimulate the generation of, a biofluid, wherein the biofluid generation unit comprises at least one sweat generating hydrogel, and a muscarinic receptor agonist; a microfluidic channel configured to transport the biofluid, wherein the microfluidic channel comprises an input end configured to receive the biofluid and an output end; a sensing module in fluid communication with the output end of the microfluidic channel and comprising one or more electrochemical aptamer-based sensors, wherein the one or more electrochemical aptamer-based sensors are functionalized with aptamers comprising attached reporters that are displaceable by a target analyte; electrodes configured to produce a potential sufficient for exchanging charged particles with the displaced reporters, or with conjugates of the displaced reporters, wherein the displaced reporters are configured to be hybridized with an enhancer to amplify a signal generated by the displaced reporters; and a processor communicatively coupled to the sensing module, the biofluid generation unit, the electrode, and a memory configured to store computer readable instructions, wherein the processor is configured to execute the computer readable instructions comprising: causing activation of the electrodes to exchange the charged particles with the displaced reporters, or with conjugates of the displaced reporters, receiving a measurement of the exchanged charged particles, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

Example 2. The device of any one of the preceding examples, but particularly Example 1, wherein the improved circadian rhythm is an improved circadian amplitude.

Example 3. The device of any one of the preceding examples, but particularly Example 1, wherein the circadian entraining factors comprise one or more of: a timing of light exposure, an intensity of light exposure, a meal timing, an exercise timing, or a sleep timing.

Example 4. The device of any one of the preceding examples, but particularly Example 1, wherein the biofluid generation unit and the microfluidic channel are detachable from the device.

Example 5. The device of any one of the preceding examples, but particularly Example 1, wherein the target analyte is one of: melatonin, cortisol, testosterone, oestrogens, growth hormone, thyroid hormones, interleukin 6, neuropeptides Y, caffeine, glucose, lactate, ammonium, zinc, magnesium, adenosine, serotonin, creatine kinase, insulin, glucagon, c-reactive protein, procalcitonin, leptin, ghrelin, thyroxin, ethanol, urea, uric acid, vitamin C, and vitamin D.

Example 6. The device of any one of the preceding examples, but particularly Example 1, wherein the computer readable instructions further comprise: analyzing a hormone variability based on the determined concentration; and updating the individualized set of circadian entraining factors based on the analyzed hormone variability.

Example 7. The device of any one of the preceding examples, but particularly Example 1, further comprising: receiving data from one or more communicatively coupled remote devices, wherein the data comprises one or more of: heart rate data, heart rate variability data, sleep data, or actigraphy data; and updating the individualized set of circadian entraining factors based on the received data.

Example 8. The device of any one of the preceding examples, but particularly Example 7, wherein the one or more communicatively coupled remote devices comprise one of: a mobile computing device, a wearable computing device, or a server.

Example 9. The device of any one of the preceding examples, but particularly Example 1, further comprising triggering generation of feedback based on data received after incorporating one or more of the individualized sets of circadian entraining factors.

Example 10. The device of any one of the preceding examples, but particularly Example 1, further comprising triggering generation of one or more reminders to employ the individualized set of circadian entraining factors.

Example 11. The device of any one of the preceding examples, but particularly Example 1, further comprising a strap configured to be removably coupled the non-invasive wearable device to a second wearable device.

Example 12. The device of any one of the preceding examples, but particularly Example 1, further comprising a housing that at least partially defines a surface configured to adhere to a skin surface of a user.

Example 13. The device of any one of the preceding examples, but particularly Example 1, wherein the non-invasive wearable device is configured to be embedded in a textile.

Example 14. The device of any one of the preceding examples, but particularly Example 1, wherein the non-invasive wearable device is configured to interface with at least a portion of a wrist of a user.

Example 15. The device of any one of the preceding examples, but particularly Example 1, wherein the non-invasive wearable device comprises a first reusable portion and a second disposable portion.

Example 16. The device of any one of the preceding examples, but particularly Example 15, wherein the second disposable portion comprises the biofluid generation unit, the microfluidic channel, and the sensing module.

Example 17. The device of any one of the preceding examples, but particularly Example 16, wherein the second disposable portion comprises an adhesive surface.

Example 18. The device of any one of the preceding examples, but particularly Example 15, wherein the first reusable portion comprises the processor, a data transmission unit, and a battery management system.

Example 19. A computer-implemented method for generating individualized circadian entraining factors, the method comprising: receiving a measurement of a target analyte in a biofluid from an electrochemical aptamer-based sensor; determining a concentration of the target analyte in the biofluid based on the received measurement; detecting a circadian rhythm based on the determined concentration; and generating an individualized set of circadian entraining factors based on the detected circadian rhythm to guide a user towards an improved circadian amplitude.

Example 20. The computer-implemented method of any one of the preceding examples, but particularly Example 19, wherein the method is executed by a processor of a mobile computing device having the method stored as computer readable instructions in a memory of the mobile computing device.

Example 21. The computer-implemented method of any one of the preceding examples, but particularly Example 19, wherein the measurement of the target analyte in the biofluid is received from a non-invasive wearable device configured to measure the target analyte.

Example 22. The computer-implemented method of any one of the preceding examples, but particularly Example 21, wherein the non-invasive wearable device comprises a sensing module comprising the electrochemical aptamer-based sensor, wherein the electrochemical aptamer-based sensor is functionalized with aptamers comprising attached reporters.

Example 23. The computer-implemented method of any one of the preceding examples, but particularly Example 22, wherein the non-invasive wearable device comprises electrodes configured to bear a potential sufficient for exchanging charged particles with the reporters displaced by the target analyte, or with conjugates of the displaced reporters, wherein: each reporter of the electrochemical aptamer-based sensor is displaceable by the target analyte, and each displaced reporter is configured to be hybridized with an enhancer to amplify a signal from the displaced reporters.

Example 24. The computer-implemented method of any one of the preceding examples, but particularly Example 19, wherein the circadian entraining factors comprise one or more of: a timing of light exposure, an intensity of light exposure, a meal timing, an exercise timing, or a sleep timing.

Example 25. The computer-implemented method of any one of the preceding examples, but particularly Example 19, wherein the target analyte is one of: melatonin, cortisol, testosterone, oestrogens, growth hormone, thyroid hormones, interleukin 6, neuropeptides Y, caffeine, glucose, lactate, ammonium, zinc, magnesium, adenosine, serotonin, creatine kinase, insulin, glucagon, c-reactive protein, procalcitonin, leptin, ghrelin, thyroxin, ethanol, urea, uric acid, vitamin C, and vitamin D.

Example 26. The computer-implemented method of any one of the preceding examples, but particularly Example 19, wherein the method further comprise: analyzing a hormone variability based on the determined concentration; and updating the individualized set of circadian entraining factors based on the analyzed hormone variability.

Example 27. The computer-implemented method of any one of the preceding examples, but particularly Example 19, further comprising: receiving data from one or more communicatively coupled remote devices, wherein the data comprises one or more of: heart rate data, heart rate variability data, sleep data, or actigraphy data; and updating the individualized set of circadian entraining factors based on the received data.

Example 28. The computer-implemented method of any one of the preceding examples, but particularly Example 25, wherein the one or more communicatively coupled remote devices comprises one of: a mobile computing device, a wearable computing device, or a server.

Example 29. The computer-implemented method of any one of the preceding examples, but particularly Example 19, further comprising triggering generation of feedback based on data received after incorporating one or more of the individualized sets of circadian entraining factors.

Example 30. The computer-implemented method of any one of the preceding examples, but particularly Example 19, further comprising triggering generation of one or more reminders to employ the individualized set of circadian entraining factors.

Example 31. A non-invasive wearable device comprising: an electrochemical, aptamer-based sensing device configured to sense and measure a target analyte in a biofluid; and a processor communicatively coupled to the electrochemical, aptamer-based sensing device, and a memory configured to store computer readable instructions, wherein the processor is configured to execute the computer readable instructions comprising: causing activation of the electrodes to exchange charged particles with displaced reporters, or with conjugates of the displaced reporters, receiving a measurement of the exchanged charged particles, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

Example 32. The non-invasive wearable device of any one of the preceding examples, but particularly 31, wherein any of the features of Examples 2-18 are incorporated.

Example 33. A non-invasive wearable device comprising: a sensing module configured to sense and measure a target analyte in a biofluid; and a processor communicatively coupled to the sensing module, and a memory configured to store computer readable instructions, wherein the processor is configured to execute the computer readable instructions comprising: receiving a measurement of the target analyte in the biofluid, determining a concentration of the target analyte in the biofluid based on the received measurement, detecting a circadian rhythm based on the determined concentration; and generating an individualized set of circadian entraining factors based on the detected circadian rhythm to guide a user towards an improved circadian rhythm.

Example 34. The non-invasive wearable device of any one of the preceding examples, but particularly 33, wherein any of the features of Examples 2-18 are incorporated.

The systems and methods described herein can be embodied and/or implemented at least in part as a machine configured to receive a computer-readable medium storing computer-readable instructions. The instructions may be executed by computer-executable components may be integrated with the system and one or more portions of the processor on the wearable device and/or computing device. The computer-readable medium can be stored on any suitable computer-readable media such as RAMs, ROMs, flash memory, EEPROMs, optical devices (e.g., CD or DVD), hard drives, floppy drives, or any suitable device. The computer-executable component may be a general or application-specific processor, but any suitable dedicated hardware or hardware/firmware combination can alternatively or additionally execute the instructions.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “sensor” may include, and is contemplated to include, a plurality of sensors. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A non-invasive wearable device comprising:
a biofluid generation unit configured to generate, or stimulate the generation of, a biofluid, wherein the biofluid generation unit comprises at least one sweat generating hydrogel, and a muscarinic receptor agonist;
a microfluidic channel configured to transport the biofluid, wherein the microfluidic channel comprises an input end configured to receive the biofluid and an output end;
a sensing module in fluid communication with the output end of the microfluidic channel and comprising one or more electrochemical aptamer-based sensors, wherein the one or more electrochemical aptamer-based sensors are functionalized with aptamers comprising attached reporters that are displaceable by a target analyte;
electrodes configured to produce a potential sufficient for exchanging charged particles with the displaced reporters, or with conjugates of the displaced reporters, wherein the displaced reporters are configured to be hybridized with an enhancer to amplify a signal generated by the displaced reporters; and
a processor communicatively coupled to the sensing module, the biofluid generation unit, the electrode, and a memory configured to store computer readable instructions, wherein the processor is configured to execute the computer readable instructions comprising:
causing activation of the electrodes to exchange the charged particles with the displaced reporters, or with conjugates of the displaced reporters,
receiving a measurement of the exchanged charged particles,
determining a concentration of the target analyte in the biofluid based on the received measurement,
detecting a circadian rhythm based on the determined concentration; and
generating an individualized set of circadian entraining factors based on the detected circadian rhythm to guide a user towards an improved circadian rhythm.

2. The device of claim 1, wherein the improved circadian rhythm is an improved circadian amplitude.

3. The device of claim 1, wherein the circadian entraining factors comprise one or more of: a timing of light exposure, an intensity of light exposure, a meal timing, an exercise timing, or a sleep timing.

4. The device of claim 1, wherein the biofluid generation unit and the microfluidic channel are detachable from the device.

5. The device of claim 1, wherein the target analyte is one of: melatonin, cortisol, testosterone, estrogens, growth hormone, thyroid hormones, interleukin 6, neuropeptides Y, caffeine, glucose, lactate, ammonium, zinc, magnesium, adenosine, serotonin, creatine kinase, insulin, glucagon, c-reactive protein, procalcitonin, leptin, ghrelin, thyroxin, ethanol, urea, uric acid, vitamin C, and vitamin D.

6. The device of claim 1, wherein the computer readable instructions further comprise:
analyzing a hormone variability based on the determined concentration; and
updating the individualized set of circadian entraining factors based on the analyzed hormone variability.

7. The device of claim 1, further comprising:
receiving data from one or more communicatively coupled remote devices, wherein the data comprises one or more of: heart rate data, heart rate variability data, sleep data, or actigraphy data; and
updating the individualized set of circadian entraining factors based on the received data.

8. The device of claim 7, wherein the one or more communicatively coupled remote devices comprise one of: a mobile computing device, a wearable computing device, or a server.

9. The device of claim 1, further comprising triggering generation of feedback based on data received after incorporating one or more of the individualized set of circadian entraining factors.

10. The device of claim 1, further comprising triggering generation of one or more reminders to employ the individualized set of circadian entraining factors.

11. The device of claim 1, further comprising a strap configured to removably couple the non-invasive wearable device to a second wearable device.

12. The device of claim 1, further comprising a housing that at least partially defines a surface configured to adhere to a skin surface of the user.

13. The device of claim 1, wherein the non-invasive wearable device is configured to be embedded in a textile.

14. The device of claim 1, wherein the non-invasive wearable device is configured to interface with at least a portion of a wrist of the user.

15. The device of claim 1, wherein the non-invasive wearable device comprises a first reusable portion and a second disposable portion.

16. The device of claim 15, wherein the second disposable portion comprises the biofluid generation unit, the microfluidic channel, and the sensing module.

17. The device of claim 16, wherein the second disposable portion comprises an adhesive surface.

18. The device of claim 15, wherein the first reusable portion comprises the processor, a data transmission unit, and a battery management system.

19-30. (canceled)

31. A non-invasive wearable device comprising:
an electrochemical, aptamer-based sensing device configured to sense and measure a target analyte in a biofluid; and
a processor communicatively coupled to the electrochemical, aptamer-based sensing device, and a memory configured to store computer readable instructions, wherein the processor is configured to execute the computer readable instructions comprising:
causing activation of an electrodes to exchange charged particles with displaced reporters, or with conjugates of the displaced reporters, receiving a measurement of the exchanged charged particles,
determining a concentration of the target analyte in the biofluid based on the received measurement,
detecting a circadian rhythm based on the determined concentration; and
generating an individualized set of circadian entraining factors based on the detected circadian rhythm to guide a user towards an improved circadian rhythm.

32. (canceled)
